(19) United States
(12) Patent Application Publication
(10) Pub. No.: US 2006/0205999 A1
(43) Pub. Date: Sep. 14, 2006

(54) Transportation of biological matter to a target site in a closed volume for transplantation purposes

(76) Inventors: Avraham Berger, Givataim (IL); Ben Zion Haim, Kiryat Ono (IL); Baruch Meirovich, Hod Hasharon (IL); Avri Hazan, Givataim (IL)

Correspondence Address: NATH & ASSOCIATES, 112 South West Street, Alexandria, VA, 22314, US

(21) Appl. No.: 10/553,266

(22) PCT Filed: Apr. 20, 2003
(86) PCT No.: PCT/IL03/00324

§ 371 Date: Oct. 11, 2005

(30) Foreign Application Priority Data
Apr. 22, 2002 (US) 10126947

(51) Int. Cl. A61B 17/43

(52) U.S. Cl. 600/34

(57) ABSTRACT

The present invention is directed toward the transportation of biological matter to a target site in a closed volume for transplantation purposes. The present invention involves provisioning WO99/18872's flattened droplet delivery apparatus with a pressure sensor for real time monitoring of the prevailing pressure in its transfer tube for feedback purposes. The present invention also includes a guide catheter for enabling the introduction of a delivery catheter to a target site in a closed volume and concurrent fluid drainage therefrom to discharge pressure. The present invention also includes a tubing set for use in a cell therapy procedure.

### FIELD OF THE INVENTION

The invention is in the field of transporting biological matter to a target site in a closed volume for transplantation purposes.

### BACKGROUND OF THE INVENTION

In commonly assigned WO99/18872 entitled “Method for Depositing a Flattened Droplet on a Surface and Apparatus Therefor, and a Pump Therefor”, the contents of which are incorporated herein by reference, there is illustrated and described a flattened droplet type IVF-ET procedure in which preferably a single embryo is accurately deposited on an embryo recipient subject's endometrium. One advantage of the flattened droplet approach is that it employs only about 0.3-2 μl culture medium to transport an embryo to a target site as opposed to 20-40 μl culture medium in the conventional IVF-ET injection approach but surprisingly, even this microvolume may increase a typical 3-4 Inches of Water uterine prevailing pressure by 1-3 Inches of Water. Another advantage of the flattened droplet approach is that an embryo is subjected to a far smaller pressure than in comparison to the conventional IVF-ET injection approach. Clinical trials have demonstrated that the flattened droplet approach leads to a considerably higher percentage of successful pregnancies in comparison to the conventional IVF-ET injection approach. However, this notwithstanding, the flattened droplet IVF-ET approach still suffers from technical faults, for example, kinking of a delivery catheter, blockage of its distal end, and the like, which in all likelihood lead to an unsuccessful IVF-ET procedure.

### SUMMARY OF THE INVENTION

The present invention is directed toward the transportation of biological matter to a target site in a closed volume for transplantation purposes. The present invention involves provisioning the aforementioned WO99/18872's flattened droplet delivery apparatus with a pressure sensor for real time monitoring of the prevailing pressure in its transfer tube. The prevailing pressure can be displayed on a monitor particularly during the actual transfer of an embryo bearing culture medium microvolume to an embryo recipient subject's endometrium such that an occurrence of one or more of the aforelisted technical faults would be immediately apparent to a trained practitioner who may then take appropriate corrective action. Pattern recognition functionality can be readily applied to a pressure waveform of the prevailing pressure within a transfer tube for detecting an occurrence of one or more of the aforelisted technical faults by virtue of each technical fault having a uniquely identifiable fault signature, thereby enabling the automatic issuance of a visual and/or aural alarm in the case of such an occurrence. To largely negate the aforesaid typical 1-3 Inches of Water prevailing uterine pressure increase during a flattened droplet type IVF-ET procedure, the delivery catheter for delivering the embryo bearing culture medium microvolume to an embryo recipient subject's uterus is preferably introduced through an extruded guide catheter designed to slidingly support the threading of the delivery catheter therethrough, and enable concurrent fluid drainage from her uterus to lower her prevailing uterine pressure.

The present invention also enables the transportation of a series of biological matter bearing flattened droplets for effecting cell based therapy procedures rather than the presently suggested injection approach as described in an on-line article entitled “Stem Cells: A primer” as retrieved from the National Institute of Health (NIH)'s website on Apr. 12, 2003 <URL:

www.nih.gov/news/stemcell/primer.htm>. Cell based therapies are now being hypothesized to treat a wide range of diseases including inter alia Parkinson's disease, diabetes, traumatic spinal cord injury, heart disease, vision and hearing loss, and the like. In this context, biological matter may be in the form of complete cells, for example, stem cells, germ cells, and the like, and also cell components, for example, DNA, RNA, and the like. It is believed that the flattened droplet approach in comparison to the conventional injection approach will be particularly advantageous for cell based therapy procedures by virtue of the fact that the aforesaid advantages of the former approach will be even more beneficial due to cell based therapy procedures requiring the transplantation of far greater quantities of biological matter than preferably a single embryo for an IVF-ET procedure, and the target sites of cell based therapy procedures are in closed volumes far smaller than a human uterus and therefore more susceptible to increases in prevailing pressures.

### BRIEF DESCRIPTION OF DRAWINGS

In order to understand the invention and to see how it may be carried out in practice, preferred embodiments will now be described, by way of non-limiting examples only, with reference to the accompanying drawings, in which:

FIG. 1 is a pictorial representation of apparatus for effecting a flattened droplet type IVF-ET procedure in accordance with the present invention;

FIG. 2 is a transverse cross section view of a guide catheter with a delivery catheter threaded therethrough for use in a flattened droplet type IVF-ET procedure;

FIGS. 3A-3F are pictorial representations showing the transport of an embryo bearing culture medium microvolume to an embryo recipient subject's endometrium during a flattened droplet type IVF-ET procedure;

FIG. 4 is a graph showing the pressure waveform of the prevailing pressure in the transfer tube of the apparatus of FIG. 1 during a flattened droplet type IVF-ET procedure together with fault signatures of three potential technical faults which may occur during such a procedure;

FIG. 5 is a pictorial representation of apparatus for transporting biological matter for a cell based therapy procedure in accordance with the present invention;

FIGS. 6A to 6C are pictorial representations showing the operation of the apparatus of FIG. 5;

FIG. 7 is a graph showing the prevailing pressure within the transfer tube of the apparatus of FIG. 5 during a cell based therapy procedure; and

FIG. 8 is a pictorial representation showing the fusing together of a number of stem cell bearing flattened droplets into a large drop.

### DESCRIPTION OF PREFERRED EMBODIMENTS

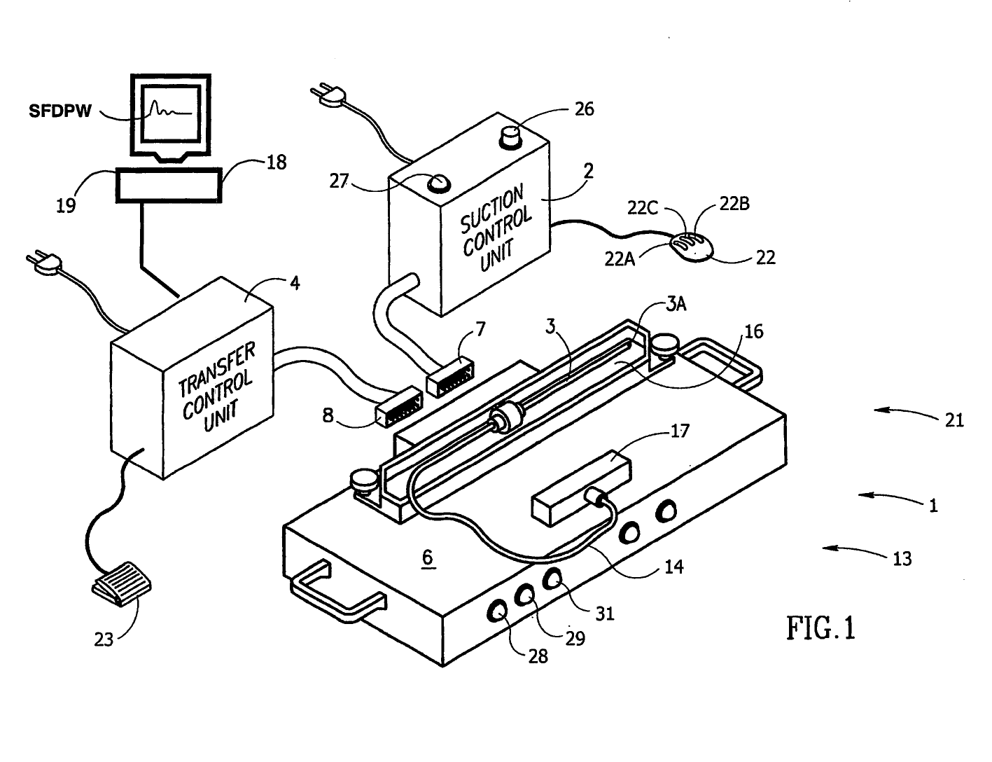

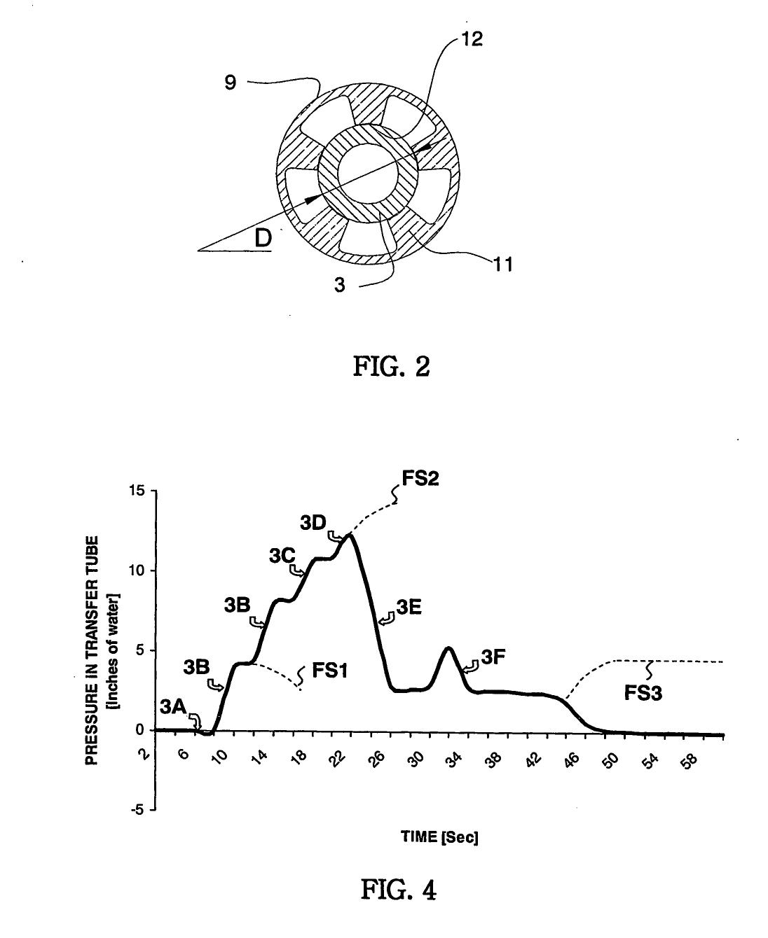

FIG. 1 shows apparatus 1 for effecting an improved flattened droplet type IVF-ET procedure. Apparatus 1 includes a suction control unit 2 typically permanently located in a laboratory for the preparation of an embryo bearing delivery catheter (constituting a narrow bore transfer tube) 3, a transfer control unit 4 typically permanently located in a treatment room where IVF-ET procedures are carried out and a portable casing 6 for consecutive connection to the suction control unit 2 and the transfer control unit 4 by means of connectors 7 and 8. Insertion of the delivery catheter 3 into an embryo recipient subject's uterus is preferably through an extruded guide catheter 9 having, say, four to eight, longitudinally directed supports 11 whose longitudinally directed curved inner facing surfaces 12 define an imaginary circular in the transverse cross section view of FIG. 2 with a diameter slightly larger than the delivery catheter's diameter D whereby the delivery catheter 3 can be readily slidingly threaded therethough. The use of the guide catheter 9 enables fluid drainage from an embryo recipient subject's uterus to avoid a significant increase in her uterine pressure which may militate against a successful IVF-ET procedure.

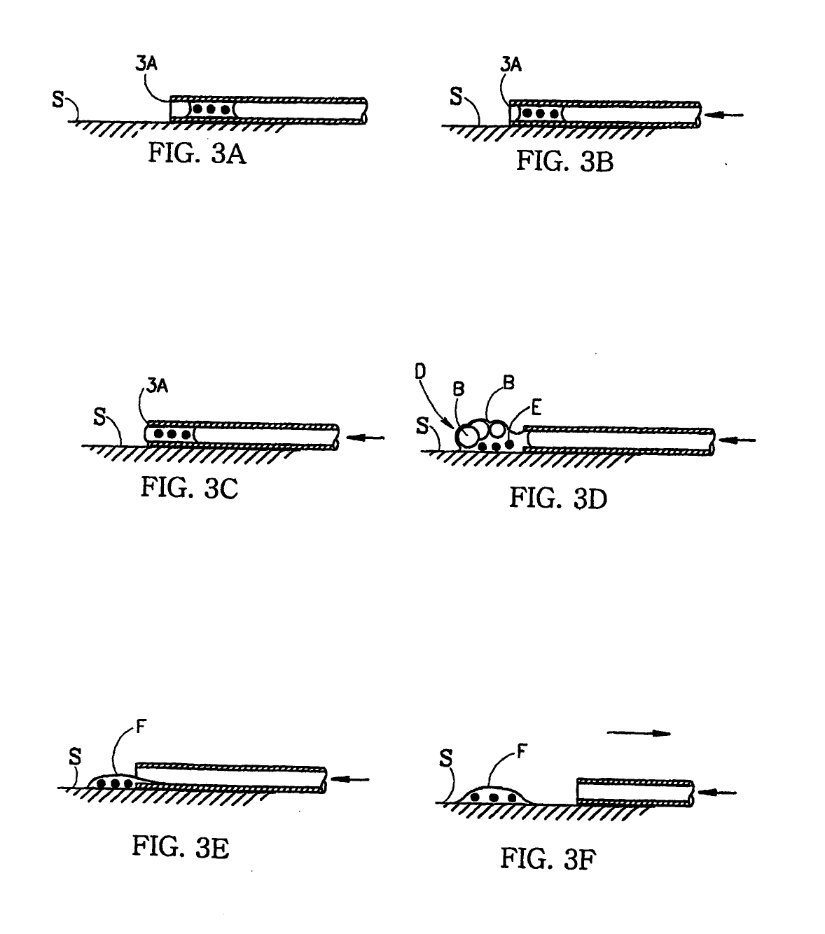

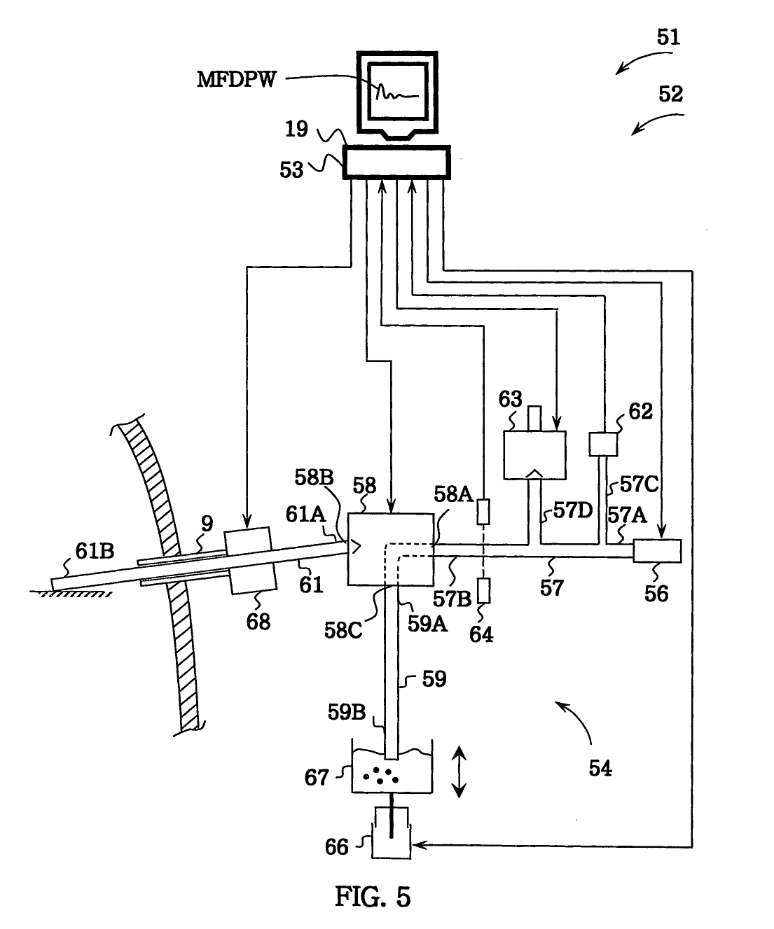

The casing 6 includes a pneumatic system 13 which is permanently connected to the delivery catheter 3 during an entire IVF-ET procedure via suitable air tubing 14, and a receptacle 16 for accommodating the delivery catheter 3 during the transport of the casing 6 from a laboratory to a treatment room. The pneumatic system 13 includes a microvolume pump (not shown), for example, as described with reference to WO99/18872's FIGS. 3-5, and a pressure sensor 17 for monitoring the prevailing pressure within the delivery catheter 3 for display as a Single Flattened Droplet Pressure Waveform (SFDPW) on a computer 18. The pressure sensor 17 is operative over 0-1 psi a pressure range and has an about ±1% pressure sensitivity. IC Sensor's Model 1210 pressure sensor would be suitable for use as pressure sensor 17. The computer 18 can be programmed with pattern recognition functionality 19 to automatically issue visual and/or aural alarms on detection of an occurrence of one or more fault signatures in the SFDPW as described hereinbelow with reference to FIG. 4.

The pneumatic system 13 is under the control of a control mechanism 21 including a control pad 22 for controlling the suction control unit 2 for initiating a user controlled suction mode to load the delivery catheter 3 with an embryo bearing culture medium microvolume and a foot pedal 23 for controlling the transfer control unit 4 for initiating a user initiated automated delivery mode for depositing an embryo bearing flattened droplet on an embryo recipient subject's endometrium. The control pad 22 has an upstroke control 22A for drawing an incoming flow of displacement gas into the pneumatic system 13 from the delivery catheter 3, a downstroke control 22B for issuing an outgoing flow of displacement gas from the pneumatic system 13 into the delivery catheter 3, and optionally a speed control 22C for controlling the flow rate of the displacement gas either from or into the delivery catheter 3. The suction control unit 2 is also provided with a reset button 26 for priming the pneumatic system 13 for a pre-suction mode of issuing an outgoing flow of displacement gas as indicated by a READY indicator light 27 prior to the loading of the delivery catheter 3. The different stages of the automatic delivery mode are indicated by a READY indicator light 28, a GO indicator light 29 and a DONE indicator light 31.

For the sake of conciseness, the loading of the delivery catheter 3 with one or more embryo(s) in accordance with the procedure described with reference to WO99/18872's FIGS. 2A-2E is not repeated here. The depositing of an embryo bearing flattened droplet on an embryo recipient subject's endometrium S is now described with reference to the steps shown in FIGS. 3A-3F and SFDPW shown in FIG. 4.

The delivery catheter's distal end 3A is laid on an embryo recipient subject's endometrium S (see FIG. 3A) whereupon a first depression on the foot pedal 23 causes the READY indicator light 28 to be lit indicating that the automatic delivery mode can be initiated. Thereafter, a second depression on the foot pedal 23 causes the GO indicator light 29 to be lit indicating that an outgoing flow of displacement gas is displacing the embryo bearing culture medium microvolume towards the distal end 3A (see FIG. 3B). The outgoing flow of displacement gas causes a concave shaped meniscus to be slowly formed which increases in size until it suddenly ruptures whereby most of the embryo bearing culture medium microvolume is discharged as a droplet D on the surface S (see FIGS. 3C and 3D). The discharge is accompanied by blowing miniscule air bubbles B into the droplet D for frothing it and thereby considerably widening its projected surface area on the embryo recipient subject's endometrium S to form the flattened droplet F whose shape is maintained by its prevailing surface tension with the embryo recipient subject's endometrium S (see FIG. 3E).

The GO indicator light 29 is then extinguished indicating that the practitioner should slightly withdraw the delivery catheter 3 so as to detach its distal end 3A from the droplet F whilst at the same time there is a slow discharge of displacement gas. Withdrawal is limited to between about 10-15 mm such that the distal end 3A still lies along the embryo recipient subject's endometrium S. And finally, a further outgoing pulse of displacement gas is provided to clean the distal end 3A of any remaining culture medium (see FIG. 3F). The DONE indicator light 31 is then lit to indicate that the delivery catheter 3 can be completely removed from the embryo recipient subject's uterus.

FIG. 4 also shows three fault signatures FS1, FS2 and FS3 superimposed on the SFDPW of technical faults which may occur during a flattened droplet type IVF-ET procedure as follows:

A fault signature FS1 appears as a pressure drop at the beginning of the SFDPW during an initial issue of outgoing flow of displacement gas to outwardly displace the embryo bearing culture medium microvolume along the delivery catheter 3. A fault signature FS1 is indicative that the pneumatic system 13 is not hermetically sealed and that suitable action be taken to hermetically seal the pneumatic system 13 before a flattened droplet type IF-ET procedure can be continued.

A fault signature FS2 appears as a pressure increase beyond a predetermined maximum pressure, say, 12 Inches of Water for an IVF-ET procedure. A fault signature FS2 is indicative of either a kink along the delivery catheter 3 or a blockage at its distal end 3A due to soft tissue or blood. A fault signature FS2 requires that a practitioner attempt to unkink the delivery catheter 3 or unblock its distal end 3A by gently manipulate the delivery catheter 3 to and fro or up and down.

A fault signature FS3 appears as a pressure increase towards the end of the SFDPW after the issue of the further outgoing pulse of displacement gas denoted 3F in FIG. 4. The pressure increase appears instead of the prevailing pressure in the delivery catheter 3 dropping down to a typical uterine prevailing pressure of about 3-4 Inches of Water. A fault signature FS3 is indicative of the back flow of a flattened droplet F possibly including the embryo E into the delivery catheter 3. A fault signature FS3 requires that a practitioner issue a second outgoing pulse of displacement gas.

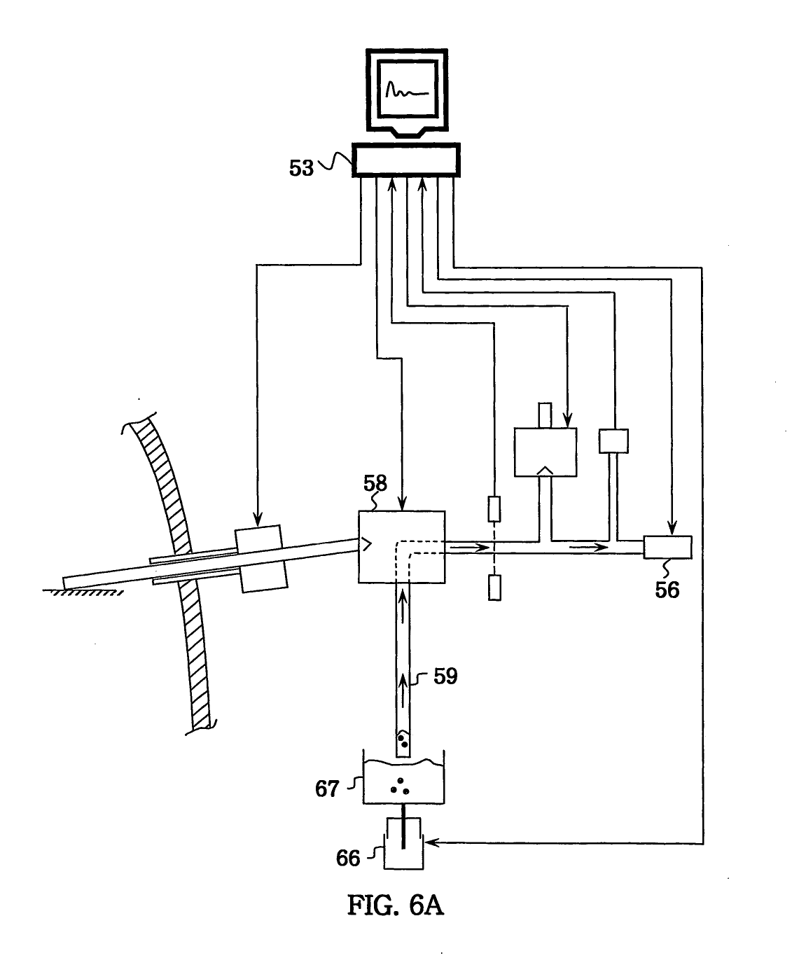

FIG. 5 shows apparatus 51 for automatically transporting a series of biological matter bearing flattened droplets to a target site for, say, a stem cell therapy procedure. The apparatus 51 includes a pneumatic system 52 under the control of a computer (constituting a control mechanism) 53 including the pattern recognition functionality 19 programmed for detecting the fault signatures FS1, FS2 and FS3, and issuing suitable alarms, and a tubing set 54 designed for single or multiple use. The pneumatic system 52 includes a microvolume pump 56, for example, as described with reference to WO99/18872's FIGS. 3-5.

The tubing set 54 includes a transfer tube 57 having a proximal end 57A, a distal end 57B, and a pair of tube segments 57C and 57D; a two-way valve 58 under the control of the computer 53 and having three ports 58A, 58B and 58C; an uptake catheter 59 having a proximal end 59A and a distal end 59B; and a delivery catheter 61 having a proximal end 61A and a distal end 61B. The prevailing pressure in the transfer tube 57 is monitored by a pressure sensor 62 hermetically connected to the tube segment 57C and connected to the computer 53 for real time feedback purposes and for displaying a Multiple Flattened Droplet Pressure Waveform (MFDPW) arising from a stem cell therapy procedure. The transfer tube 57 can be selectively vented to atmospheric pressure P₀ by a normally closed (NC) venting valve 63 hermetically connected to the tube segment 57D and under the control of the computer 53.

The computer 53 also receives closed loop feedback from an electro-optical culture medium microvolume detection device 64 for detecting the presence of a stem cell bearing culture medium microvolume within a predetermined segment of the transfer tube 54. The computer 53 also controls a lifting device 66 for controlling the height of a source of stem cells 67 for selectively immersing the uptake catheter's distal end 59B therein, and a micromanipulator 68 for controlling the location of the delivery catheter's distal end 61B.

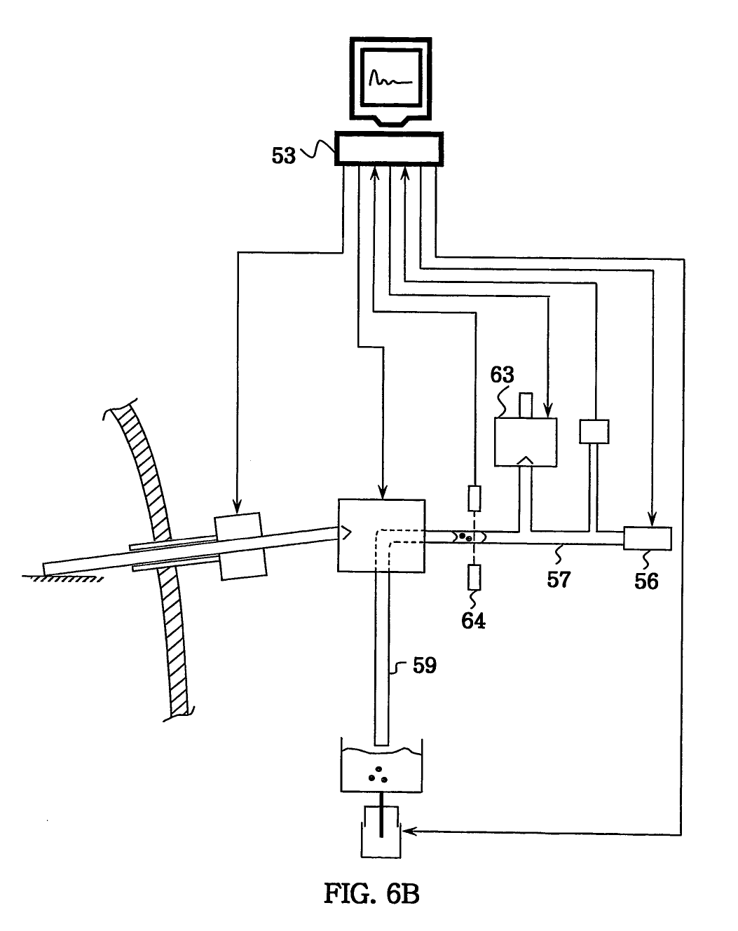

Operation of apparatus 51 for transplanting a single stem cell bearing flattened droplet at the target site is now described with reference to FIGS. 6A-6C is as follows:

The computer 53 sets the 2-way valve 58 to connect the uptake catheter 59 to the microvolume pump 56, operates the lifting device 66 to lift the stem cell source 67 to immerse the uptake catheter's distal end 59B therein, controls the microvolume pump 56 for drawing an incoming flow of displacement gas thereinto so as to aspirate a stem cell bearing culture medium microvolume into the uptake catheter 59, and then operates the lifting device 66 a second time to lower the stem cell source 67 to remove the distal end 59B therefrom (see FIG. 6A).

The computer 53 continues to operate the microvolume pump 56 to draw an incoming flow of displacement gas thereinto to aspirate the stem cell bearing culture medium microvolume into the transfer tube 57 towards its proximal end 57A. Upon its reaching the culture medium microvolume detection device 64, the culture medium microvolume detection device 64 sends a signal to the computer 53 which interrupts the operation of the microvolume pump 56 and opens the NC venting valve 63 to stop the inward movement of the stem cell bearing culture medium microvolume (see FIG. 6B).

The computer 53 closes the NC venting valve 63, sets the 2-way valve 58 to connect the transfer tube 57 to the delivery catheter 61, and operates the microvolume pump 56 to issue an outgoing flow of displacement gas to outwardly displace the stem cell bearing culture medium microvolume towards the distal end 61B (see FIG. 6C). The computer 53 operates the microvolume pump 56 to deposit a stem cell bearing flattened droplet at the target site in a similar manner as described hereinabove with respect to the flattened droplet type IVF-ET procedure.

The computer 53 operates the micromanipulator 68 to withdraw the delivery catheter's distal end 61B by about 0.5-3 mm to detach a stem cell bearing flattened droplet therefrom. The computer 53 also operates the micromanipulator 68 to gently manipulate the delivery catheter 61 on detection of a fault signature FS2 and the microvolume pump 56 on detection of a fault signature FS3.

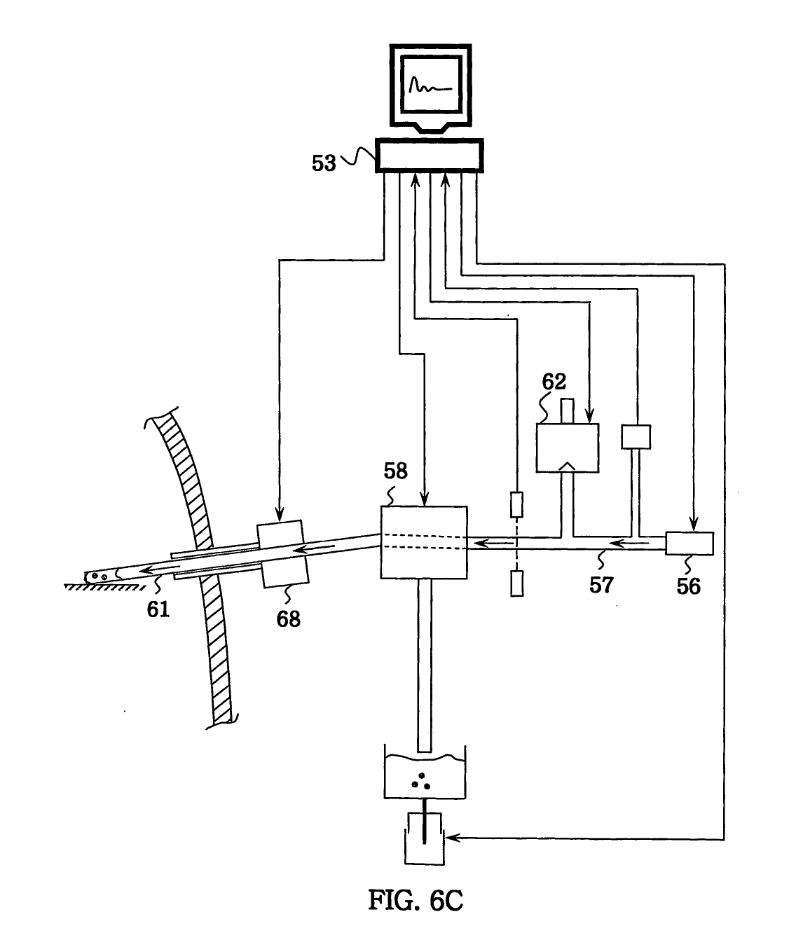

FIG. 7 shows a Multiple Flattened Droplet Pressure Waveform (MFDPW) corresponding to the transportation of successive stem cell bearing flattened droplets to a target site. The MFDPW has a stepped appearance reflecting the fact that each transplantation of a stem cell flattened droplet at the target site increases the volume thereat which in turn increases the prevailing pressure in the transfer tube 57 required to deposit the next flattened droplet. The MFDPW is effectively constituted by a series of SFDPWs (see FIG. 4) except with an additional sub-atmospheric pressure segment between each pair of consecutive SFDPWs. The sub-atmospheric pressure segments each correspond to the loading of the uptake catheter 59 with a stem cell culture medium microvolume and thereafter its inward displacement to the transfer tube 57.

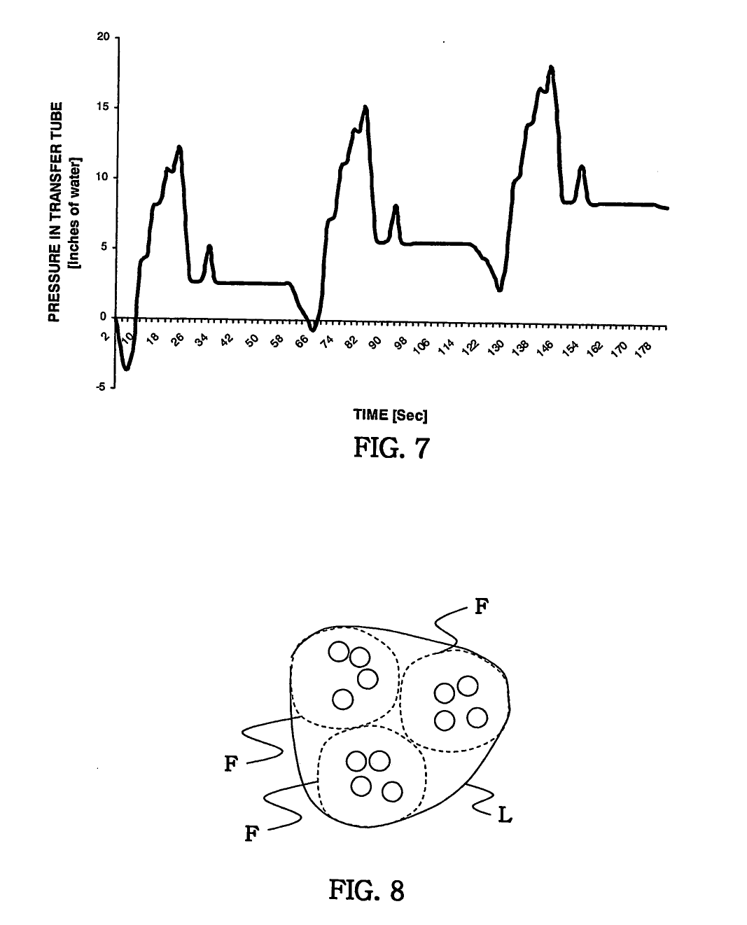

FIG. 8 shows that stem cell bearing flattened droplets D tend to fuse into a single large drop L if deposited sufficiently adjacent to one another at the target site.

Various modifications and changes may be made in the configuration described hereinabove that come within the spirit of the invention. The invention embraces all such changes and modifications coming within the scope of the appended claims.

1. Method for transporting biological matter to a target site in a closed volume for transplantation purposes, the method comprising the steps of:
(a) providing a narrow bore transfer tube having a proximal end and a distal end and containing a biological matter bearing culture medium microvolume, the proximal end connected to a pneumatic system adapted for issuing an outgoing flow of displacement gas into the transfer tube and drawing an incoming flow of displacement gas therefrom for respectively displacing the biological matter bearing culture medium microvolume towards the distal end and away therefrom;
(b) issuing an outgoing flow of displacement gas for displacing substantially the entire biological matter bearing culture medium microvolume along the transfer tube for depositing as a biological matter bearing droplet on a surface at the target site and controllably blowing miniscule air bubbles into the biological matter bearing droplet for flattening same on the surface; and
(c) monitoring the prevailing pressure within the transfer tube during step (b) for providing real time information with respect to the advancement of the biological matter bearing culture medium microvolume along the transfer tube and its successful deposit as a biological matter bearing droplet at the target site.

2. Method according to claim 1 wherein step (c) includes graphically displaying the prevailing pressure in the transfer tube on a monitor.

3. Method according to claim 1 wherein step (c) includes automatically detecting a fault signature in a pressure waveform acquired during step (b).

4. Method according to claim 3 wherein step (c) includes automatically detecting at least one of the following fault signatures in the pressure waveform: a fault signature FS1 indicative of a pressure drop during an initial outgoing flow of displacement gas into the transfer tube to displace the biological matter bearing culture medium microvolume towards its distal end; a fault signature FS2 indicative of a pressure increase beyond a predetermined maximum pressure in the transfer tube; and a fault signature FS3 indicative of a pressure increase after an additional outgoing pulse of displacement gas to clean the transfer tube's distal end of any remaining culture medium microvolume.

5. Method according to claim 3 and further comprising step (d) of automatically issuing a visual and/or aural alarm on detection of a fault signature in the pressure waveform.

6. Method according to claim 1 and further comprising the steps of:
(e) providing a 2-way valve for selectively connecting the transfer tube to either an uptake catheter with a distal end selectively immersible in a biological matter source or a delivery catheter with a distal end located at the target site;
(f) providing a venting valve for selectively venting the transfer tube's proximal end to atmospheric pressure;
(g) providing a culture medium microvolume detection device for detecting the presence of a culture medium microvolume at a predetermined section along the transfer tube;
(h) selectively immersing the uptake catheter's distal end into the biological matter source and drawing an incoming flow of displacement gas for aspirating a biological matter bearing culture medium microvolume into the uptake catheter;
(i) removing the uptake catheter's distal end from the biological matter source and drawing an incoming flow of displacement gas for aspirating the biological matter bearing culture medium microvolume into the transfer tube;
(j) venting the transfer tube on detection of the presence of the biological matter bearing culture medium microvolume therein;
(k) operating the 2-way valve to connect the transfer tube to the delivery catheter and continuing with step (b); and
(l) repeating steps (h) to (k) to transport a series of biological matter bearing flattened droplets to the target site.

7. Method according to claim 6 wherein step (h) includes lifting the biological matter source relative to the uptake catheter's distal end for immersing same therein and step (i) includes lowering the biological matter source for removing the uptake catheter's distal end therefrom.

8. Method according to claim 6 and further comprising step (m) of providing a micromanipulator for controlling the location of the delivery tube's distal end at the target site.

9. Method according to claim 8 and further comprising step (n) of operating the micromanipulator to gently manipulate the delivery catheter on detection of a fault signature FS2 in the pressure waveform.

10. Method according to claim 6 and further comprising step (o) of operating the pneumatic system to issue a second outgoing pulse of displacement gas on detection of a fault signature FS3 in the pressure waveform.

11. Apparatus for transporting biological matter to a target site in a closed volume for transplantation purposes, the apparatus for use with a narrow bore transfer tube having a proximal end and a distal end and containing a biological matter bearing culture medium microvolume, the apparatus comprising:
(a) a pneumatic system connected to the transfer tube's proximal end and adapted for issuing an outgoing flow of displacement gas into the transfer tube and drawing an incoming flow of displacement gas therefrom for respectively displacing the biological matter bearing culture medium microvolume towards the transfer tube's distal end and away therefrom;
(b) a control mechanism for controlling the pneumatic system for issuing an outgoing flow of displacement gas for discharging substantially the entire biological matter bearing culture medium microvolume as a biological matter bearing droplet on a surface at the target site and controllably blowing miniscule air bubbles into the biological matter bearing droplet for flattening same on the surface; and
(c) a pressure sensor for monitoring the prevailing pressure within the transfer tube for providing real time information with respect to the advancement of the biological matter bearing culture medium microvolume along the transfer tube and its successful deposit as a biological matter bearing droplet at the target site.

12. Apparatus according to claim 11 wherein the prevailing pressure in the transfer tube is graphically displayed on a monitor.

13. Apparatus according to claim 11 and further comprising pattern recognition functionality for automatically detecting a fault signature in a pressure waveform monitored during the displacement of substantially the entire biological matter bearing culture medium microvolume along the transfer tube and its depositing on the surface at the target site.

14. Apparatus according to claim 13 wherein the pattern recognition functionality is capable of automatically detecting at least one of the following fault signatures in the pressure waveform: a fault signature FS1 indicative of a pressure drop during an initial outgoing flow of displacement gas into the transfer tube to displace the biological matter bearing culture medium microvolume towards its distal end; a fault signature FS2 indicative of a pressure increase beyond a predetermined maximum pressure in the transfer tube; and a fault signature FS3 indicative of a pressure increase after an additional outgoing pulse of displacement gas for cleaning the transfer tube's distal end.

15. Apparatus according to claim 13 wherein the pattern recognition functionality is capable of automatically issuing a visual and/or aural alarm on detection of a fault signature in the pressure waveform.

16. Apparatus according to claim 11 and further comprising:
(d) a 2-way valve for selectively connecting the transfer tube to either an uptake catheter with a distal end selectively immersible in a biological matter source or a delivery catheter with a distal end located at the target site;
(e) a venting valve for selectively venting the transfer tube's proximal end to atmospheric pressure;
(f) a culture medium microvolume detection device for detecting the presence of a culture medium microvolume at a predetermined section along the transfer tube;
wherein the control mechanism is programmed to:
i) selectively immerse the uptake catheter's distal end into the biological matter source and draw an incoming flow of displacement gas for aspirating a biological matter bearing culture medium microvolume into the uptake catheter;
ii) remove the uptake catheter's distal end from the biological matter source and draw an incoming flow of displacement gas for aspirating the biological matter bearing culture medium microvolume into the transfer tube;
iii) vent the transfer tube on detection of the presence of the biological matter bearing culture medium microvolume therein;
iv) operate the 2-way valve to connect the transfer tube to the delivery catheter;
v) control the pneumatic system for issuing an outgoing flow of displacement gas for discharging substantially the entire biological matter bearing culture medium microvolume as a biological matter bearing droplet on a surface at the target site and controllably blowing miniscule air bubbles into the biological matter bearing droplet for flattening same on the surface; and
vi) repeat steps (i) to (v) to transport a series of biological matter bearing flattened droplets to the target site.

17. Apparatus according to claim 16 and further comprising a lifting device for lifting the biological matter source relative to the uptake catheter's distal end for immersing same therein and lowering the biological matter source for removing the uptake catheter's distal end therefrom.

18. Apparatus according to claim 16 and further comprising a micromanipulator for controlling the location of the delivery tube's distal end at the target site.

19. Apparatus according to claim 18 wherein the control mechanism operates the micromanipulator to gently manipulate the delivery catheter on detection of a fault signature FS2 in the pressure waveform.

20. Apparatus according to claim 16 wherein the control mechanism operates the pneumatic system to issue a second outgoing pulse of displacement gas on detection of a fault signature FS3 in the pressure waveform.

21. A guide catheter for use with a delivery catheter having an external diameter D, the guide catheter having a longitudinally directed lumen with a multitude of longitudinally directed supports with longitudinally directed inner facing surfaces for slidingly supported the threading of the delivery catheter through the guide catheter whereby the longitudinally directed supports assume the appearance of the spokes of a wheel in a transverse cross sectional view of the guide catheter with the delivery catheter threaded therethrough.

22. The catheter according to claim 21 wherein the inner facing surfaces are curved so as to define an imaginary circle having a diameter slightly greater than the delivery catheter's external diameter D.

23. For use with a pneumatic system capable of issuing an outgoing flow of displacement gas and drawing an incoming flow of displacement gas, and a biological matter source, a tubing set for transporting the biological matter from the biological matter source to a target site in a closed volume for transplantation purposes, the tubing set comprising a 2-way valve having three ports, a transfer tube having a proximal end connected to the pneumatic system and a distal end connected to a first port of the 2-way valve's three ports, an uptake catheter having a proximal end connected to a second port of the 2-way valve's three ports and a distal end selectively immersible in the biological matter source, and a delivery catheter having a proximal end connected to the third port of the 2-way valve's three ports and a distal end for location at the target site.

24. The tubing set according to claim 23 wherein the transfer tube has a tube segment adapted for being hermetically connected to a pressure sensor for monitoring the prevailing pressure in the transfer tube.

25. The tubing set according to claim 23 wherein the transfer tube has a tube segment adapted for being hermetically connected to a venting valve for venting the transfer tube to atmospheric pressure.
